(19) United States
(12) Patent Application Publication
(10) Pub. No.: US 2020/0129172 A1
(43) Pub. Date: Apr. 30, 2020

(54) COMMUNICATING SMART SUTURES WITH TENSION FEEDBACK MECHANISM

(71) Applicant: ADVANCED SUTURE, INC., Dorado, PR (US)

(72) Inventors: Gregory A. Dumanian, Chicago, IL (US); Adom Dumanian, San Juan, PR (US)

(21) Appl. No.: 16/173,646

(22) Filed: Oct. 29, 2018

(51) Int. Cl. A61B 17/04; A61B 17/88

(52) U.S. Cl. CPC A61B 17/0469; A61B 17/8869; A61B 17/0483; A61B 2017/0496; A61B 17/0401; A61B 17/0487

(57) ABSTRACT

A medical device includes an elongated suture having a tension indicator carried by the elongated suture. The tension indicator signals over-tensioning of the elongated suture when a tensile load applied along an axial direction of the elongated suture exceeds a predetermined tensile load, thereby preventing over-tensioning of the suture and collapsing of the suture. Advantageously, the tension indicator serves as an integrated feedback device that provides instantaneous feedback when tensioning a suture, rather than having to use an external measuring device to determine the amount of tensile load applied to the suture.

### FIELD OF THE DISCLOSURE

The present disclosure is directed to sutures having structural characteristics that indicate over-tensioning and methods of use thereof.

### BACKGROUND

One of the foundations of surgery is the use of sutures to re-appose soft tissue, i.e., to hold tissue in a desired configuration until it can heal. In principle, suturing constitutes introducing a high tensile foreign construct (looped suture) into separate pieces of tissue in order to hold those pieces in close proximity until scar formation can occur, establishing continuity and strength between tissues. Sutures initially provide the full strength of the repair, but then become secondarily reinforcing or redundant as the tissue heals. The time until tissue healing reaches its maximal strength and is dependent on suture for approximation, therefore, is a period of marked susceptibility to failure of the repair due to forces naturally acting to pull the tissues apart.

Conventional sutures provide a circular or single-point cross-sectional profile extended over the length of the suture material. Such a suture has the great benefit of radial symmetry, which eliminates directional orientation, allowing the user (e.g., physician, surgeon, medic, etc.) to not have to worry about orienting the suture during use. However, a considerable disadvantage of the currently used single-point cross-section is that it does not effectively distribute force, and actively concentrates force at a geometric point (e.g., the point at the leading edge of the circle) creating a sharp edge in the axial dimension. Under these conditions, the tissue is continuously exposed to tension, increasing the likelihood that stress concentration at a geometric point or sharp edge will cut through the tissue.

Conventional sutures are static unchanging devices with a particular shape and feel. They are solid, with minimal ductile properties of elongation, as this property would potentially permit tissues under tension to separate improperly causing failure of the operation. While surgeons get tactile feedback from all instruments, needles, sutures, and other devices, these objects do not communicate with the surgeon in real time as to the characteristics of their use.

One large problem in surgery is sutures abruptly slicing through tissues due to over-tensioning. Indeed, studies of abdominal wall closures demonstrate that the majority of acute dehiscences occur in the early post-operative period before full healing can occur. These surgical failures have been blamed on poor suture placement, suture composition, patient issues such as smoking and obesity, and defects in cellular and extracellular matrices. Clinical experience in examining the cause of these surgical failures reveals that in the majority of cases the cause is tearing of the tissue around the suture, or from another perspective, intact stronger suture cutting through weaker tissue. Mechanical analysis of the suture construct holding tissue together shows that a fundamental problem with current suture design is stress concentration at the suture puncture points through the tissue. That is, as forces act to pull tissues apart, rather than stress being more evenly distributed throughout the repair, it is instead concentrated at each point where the suture pierces through the tissue. The results are twofold: (1) constant stress at suture puncture points causes sliding of tissue around suture and enlargement of the holes, leading to loosening of the repair and an impairment of wound healing, and (2) at every puncture point where the stress concentration exceeds the mechanical strength of the tissue, the suture slices through the tissue causing surgical dehiscence. In addition, high pressure on the tissue created during tightening of the surgical knot can lead to local tissue dysfunction, irritation, inflammation, infection, reduced perfusion, and in the worst case tissue necrosis. This tissue necrosis found within the suture loop is one additional factor of eventual surgical failure.

Sutures used to close the abdominal wall have high failure rates as demonstrated by the outcome of hernia formation, which represents a slow tearing of sutures through tissues overtime. If sutures held tissues in apposition perfectly, then the newly joined surfaces would never separate. Incisional hernias after laparotomy are a common problem, with the occurrence rate being between 11-23%. The failure rate of sutures used to repair incisional hernias is as high as 54%. This is a sizeable and costly clinical problem, with approximately 90,000 post-operative hernia repairs performed annually in the United States.

There has been no commercial solution to the aforementioned problems with conventional sutures. Rather, thinner sutures continue to be preferred because it is commonly thought that a smaller diameter may minimize tissue injury. However, the small cross-sectional diameter in fact increases the local forces applied to the tissue, thereby increasing suture pull-through and eventual surgical failure.

Tension applied by sutures onto tissue is important and has been studied in the laboratory, but without clear advancement in knowledge of improvements on the issue. “In another experiment, surgeons were asked to apply suture tension just ‘adequate’ for fascial closure using a ‘knot-trainer.’ 17 surgeons with a mean surgical experience of 86.7 months applied a tension of 2.8 N 1/− 1.13 N (mean+/−SD) to close a mock fascia, almost three times the tension found necessary in our experiment and with a rather high standard deviation of roughly 40% of mean. During our literature search, one study comparing the applied suture tension was found. In this study, the variation of initial suture tension ranged from 0.7 and 5.9 N although the aim of the researches was to place all sutures with the same tension. As a consequence, it may be speculated that applied suture tension during wound closure is variable and far beyond standardization and control.” Schachtrupp A, Wetter O, Hoer J. An implantable sensor device measuring suture tension dynamics: results of development and experimental work. Hernia 2016; 20: 601-606.

External devices have been created to gauge suture tension during the act of sewing by the surgeon, illustrating the surgical need to improve would healing and decrease forces by which sutures slice tissue. These devices are located between the surgeon and the suture, typically with strain gauges to allow passage of the suture for placement by the surgeon while still holding the suture with slip-gages to assess force applied. Horeman T, Meijer E0j, Harlaar J J, Lange J F, van den Dobbelsteen J J. Force sensing in surgical sutures. PLOS One 2013; 8: e84466.

### GENERAL DESCRIPTION

In contrast to conventional sutures that do not change during their application, the present disclosure is directed to smart sutures that communicate with the surgeon to indicate over-tensioning and/or appropriate tensioning of the suture during use. In one form, smart sutures with a standard linear shape provide instantaneous feedback without the need of an external device to determine the tensile load applied to the suture while the suture was being tensioned. The tensile indicator signals over-tensioning in a variety of ways. In particular, the tensile indicator may signal over-tensioning via a tactile alert, a visual alert, or an auditory alarm.

In some forms, the suture has a non-standard shape, including, for example, an open porous outer mesh wall. The insides of this cylindrical suture are not defined, and may be hollow, filled with a special tension-alerting material, honeycombed, or laced with filaments. This non-standard shaped suture may have interposed segments or regions with standard linear components, with the smart communicating segment being only a portion of the suture.

In some forms, the mesh outer wall is equipped with anti-roping elements that are tuned to a particular tension. The suture communicates with the surgeon via a change in appearance of the outer shape of the mesh pores, via a color change of the filaments, or a noise elicited by the filaments upon the reaching of a certain tension. The same suture may have segments of anti-roping elements tuned to different tensions, and so the same suture would give the surgeon feedback not of a threshold of tension, but rather to the actual tension applied during sewing.

In some forms, the suture demonstrates a first threshold of tension at a first end of the suture that is different than a second threshold of tension in the middle of the suture, and different still from a third threshold of tension demonstrated by the suture at a second end of the suture spaced away from the first end.

In some forms, the suture demonstrates tension in a non-radially symmetrical manner, where a first half of the suture is tuned to a first tension, and a second half of the suture is tuned to a second tension. While the surgeon sews, if one indicator of the suture is activated while the other is not, then the tension applied to the tissues by the suture is in a range between the first tension and the second tension. The suture would not be limited to just two tensions, but a single non-radially symmetric suture could be manufactured with multiple tension indicators.

In some forms, the suture is programmed to loosen over time, such as with the dissolution of particular filaments or fibers. In some forms, the suture is programmed to tighten over time, such as with contractions of filaments or fibers. The latter is to counteract the standard tendency of sutures to loosen immediately after placement due to suture pull-through (as per Schachtrupp).

In some forms, the suture also discourages a “foreign body response” of inflammation and fibrotic tissue formation about a suture by utilizing a macroporous structure that is also advantageously equipped with anti-roping elements. The macroporous structure seeks to minimize the foreign body response to the suture, while the anti-roping elements facilitate maintenance of the desired structural configuration of the suture when exposed to axial tensile loads, e.g., while the suture is being threaded into soft tissue. These anti-roping elements, however, do not prevent the suture from flattening with lateral loading.

“Roping” is a phenomenon in the weaving industry whereby woven, knitted, or braided mesh materials tend to elongate under tension. This elongation can cause the various elements that make up the mesh material to collapse relative to each other and thereby reduce (e.g., close) the size of the pores disposed in the mesh. As such, the “anti-roping” elements of the present disclosure advantageously resist this elongation of the mesh suture and collapsing of the pores when the suture experiences axial tensile loads. By maintaining the desired structural configuration of the mesh suture during and after threading into soft tissue, the outer wall pores remain appropriately sized to facilitate tissue integration and/or prevent suture pull through.

In one form, the mesh outer wall with the anti-roping elements acts as a spring, reflecting the linear tension applied by losing its porosity, and springing back to its unstressed shape when the tension is released. The change in the pore sizes gives the surgeon immediate visual feedback of the tension applied at that moment. The anti-roping elements can be titrated to allow the spring function to be chosen by the surgeon—i.e., stronger anti-roping elements to permit higher tensions before losing porosity, and less strong anti-roping elements for lower tension closures. Current standard sutures are static in appearance and, therefore, do not give the surgeon any visual feedback when over-tightened.

Other textile materials deform under tension including planar meshes used in abdominal wall reconstruction, and urethral slings used in stress urethral incontinence procedures. However, unlike sutures, these devices can not be adjusted or visualized once deployed. For sutures, in comparison, there are multiple loops of suture placed by the surgeon to appose tissue, and the suture communicating function happens with each throw. There is also a length of implant between the tissue and the needle driver—the clamp that holds the needle and is in turn wielded by the surgeon. The communicating aspect of the suture can be physically visualized within the tissues, and between the tissue and the needle driver.

### BRIEF DESCRIPTION OF THE DRAWINGS

FIG. 1 is a perspective view of a suture constructed in accordance with the present disclosure, and including an example tension indicator.

FIG. 2 is a detailed view of the mesh wall of the suture of FIG. 1 including an example tension indicator constructed in accordance with the present disclosure.

FIG. 3 is a detailed view of the mesh wall of the suture of FIG. 1 including another example tension indicator constructed in accordance with the present disclosure.

FIG. 4 is a detailed view of the mesh wall of the suture of FIG. 1 including yet another example tension indicator constructed in accordance with the present disclosure.

FIG. 4A-1 is a detailed view of the example tension indicator of FIG. 4 encompassed by circle A in FIG. 4.

FIG. 4A-2 is a detailed view of another example tension indicator of FIG. 4 encompassed by circle A in FIG. 4.

FIG. 5 is detailed view of the mesh wall of the suture of FIG. 1 including another example of a tension indicator constructed in accordance with the present disclosure.

FIG. 6 is a detailed view of another mesh wall of the suture of FIG. 1 including yet another example of a tension indicator constructed in accordance with the present disclosure.

FIG. 7 is a detailed view of the mesh wall of the suture of FIG. 6 in a tensioned state.

FIG. 8 is a detailed view of the mesh wall of the suture of FIG. 6 in a further tensioned state.

### DETAILED DESCRIPTION

The present disclosure provides a medical suture with a tension indicator carried by the suture itself for providing a visual, audible, olfactory, and/or tactile signal to a surgeon, for example, during suturing. In some versions, the medical suture has a macroporous construct that advantageously promotes neovascularization and normal tissue in-growth and integration subsequent to introduction into the body. Additionally, the disclosed medical suture can include anti-roping elements affixed to the macroporous material for resisting elongation and collapsing of pore size under tensile loads.

Additionally, the present disclosure provides various sutures with increased surface area and/or tissue integrative properties and methods of use and manufacture thereof. In particular, provided herein are sutures with cross-section profiles and other structural characteristics that strengthen closure, prevent suture pull-through, and/or resist infection, and methods of use thereof. In some examples, sutures are provided that strengthen closure, prevent suture pull-through, and/or resist infection by, for example: (1) having a cross sectional profile that reduces pressure at suture points, (2) having a structural composition that allows tissue in-growth into the suture, or both (1) and (2). The present disclosure is not limited by any specific means for achieving the desired ends.

Sutures typically exhibit a cross-sectional profile with radial symmetry or substantially radial symmetry. As used herein, the term “substantially radial symmetry” refers to a shape (e.g., cross-sectional profile) that approximates radial symmetry. In particular, a shape that has dimensions that are within 10% error of a shape exhibiting precise radial symmetry is substantially radially symmetric for the purpose of this disclosure. For example, an oval that is 1.1 millimeter (“mm”) high and 1.0 mm wide is substantially radially symmetric. In some examples, however, the present disclosure provides sutures that lack radial symmetry and/or substantial radial symmetry.

The sutures disclosed herein may reduce suture stress concentration at puncture points. In particular, the disclosed sutures may distribute forces toward the inner surface of the puncture hole to reduce suture stress concentration at puncture points. In some examples, suture stress concentration at puncture points may be reduced by providing sutures with cross-sectional shapes that reduce tension against the tissue and the likelihood of tissue tear at the puncture site. For example, the sutures can have a flat or elliptical cross-sectional shape. In such examples, the flat or elliptical sutures can form a ribbon-like conformation.

The disclosed sutures may reduce suture stress concentration by increasing the surface area over which force can be distributed. In particular, in some examples, the suture can have a flat or generally rounded plane rather than presenting a sharp point or line of suture to tissue, as is the case with traditional sutures. Such features increases the surface area over which force can be distributed. In some examples, one cross-sectional dimension of the suture is greater than the orthogonal cross-sectional dimension.

The use of the example sutures described herein reduces the rates of surgical dehiscence in all tissues (e.g., hernia repairs, etc.). In some embodiments, sutures are provided with cross-sectional profiles that provide optimal levels of strength, flexibility, compliance, macroporosity, and/or durability while decreasing the likelihood of suture pull-through. In some embodiments, sutures are provided with sizes or shapes to enlarge the suture/tissue interface of each suture/tissue contact point, thereby distributing force over a greater area.

The sutures of the present disclosure may also provide increased initial closure strength. For example, the disclosed sutures can provide at least a 10% increase in initial closure strength. As used herein, “initial closure strength” refers to the strength of the closure (e.g., resistance to opening), prior to strengthening of the closure by the healing or scarring processes. In some embodiments, the increased initial closure strength is due to mechanical distribution of forces across a larger load-bearing surface area that reduces micromotion and susceptibility to pull through.

The disclosed sutures may also increase the rate of achieving tissue strength from healing of tissue across the opening, from ingrowth of tissue into the integrative (porous) design of the suture, etc. In some examples, sutures of the present disclosure provide at least a 10% increase in rate of achieving tissue strength. Additionally, the increased rate of return of tissue strength across the opening can further increase the load bearing surface area, thereby promoting tissue stability and decreased susceptibility to pull through, in some examples.

The disclosed sutures may also establish closure strength earlier in the healing process when the closure is most susceptible to rupture. In some examples, the sutures can reduce the time to establish closure strength by at least a 10%. In particular, closure strength may be established earlier in the healing process due to greater initial closure strength and/or greater rate of achieving tissue strength. In some examples, sutures of the present disclosure provide at least a 10% increase in final closure strength.

Further, the strength of fully healed closure is created not only at the interface between the two apposed tissue surfaces, as is the case with conventional suture closures, but also may be created along the total surface area of the integrated suture. In such examples, tissue integration into the suture decreases the rate of suture abscesses and/or infections that otherwise occur with solid foreign materials of the same size. For example, the disclosed sutures can provide at least a 10% reduction in suture abscesses and/or infection. In some embodiments, sutures provide at least a 10% reduction in suture pull-through (e.g. through tissue (e.g., epidermal tissue, peritoneum, adipose tissue, cardiac tissue, or any other tissue in need of suturing), or through control substance (e.g., ballistic gel)).

The disclosed sutures are provided with any suitable cross-section profile or shape that provides reduced stress at the tissue puncture site, point of contact with tissue, and/or closure site. In some examples, sutures have cross-sectional dimensions (e.g., width and/or depth) between 0.1 mm and 1 cm. In some embodiments, the suture dimensions that minimize pull-through and/or provide maximum load are utilized. In some embodiments, optimal suture dimensions are empirically determined for a given tissue and suture material. In some embodiments, one or both cross-sectional dimensions of a suture are the same as the cross-sectional dimensions of a traditional suture. In some embodiments, a suture comprises the same cross-sectional area as a traditional suture, but with different shape and/or dimensions. In some embodiments, a suture comprises a greater cross-sectional area than a traditional suture. In some embodiments, a suture cross-section provides a shaped leading edge (e.g., convex) that evenly distributes force along a segment of tissue, rather than focusing it at a single point. In some embodiments, shaped sutures prevent pull-through by distributing forces across the tissue rather than focusing them at a single point. In some embodiments, sutures prevent pull-through by providing a broader cross-section that is more difficult to pull through tissue.

Sutures provided herein may include any suitable cross-sectional shape that provides the desired qualities and characteristics. In some embodiments, suture cross-sectional shape provides enhanced and/or enlarged leading edge surface distance and/or area to reduce localized pressure on tissue. Suture cross-sectional shape may be an ellipse, half-ellipse, half-circle, gibbous, rectangle, square, crescent, pentagon, hexagon, concave ribbon, convex ribbon, H-beam, I-beam, dumbbell, etc. However, the suture cross-sectional profile may include any combination of curves, lines, corners, bends, etc. to achieve a desired shape.

In some examples, an edge of the suture, which comes into contact with the tissue and/or places pressure against the tissue is broader than one or more other suture dimensions. Additionally, the edge of the suture, which comes into contact with the tissue and/or place pressure against the tissue is shaped to evenly distribute forces across the region of contact.

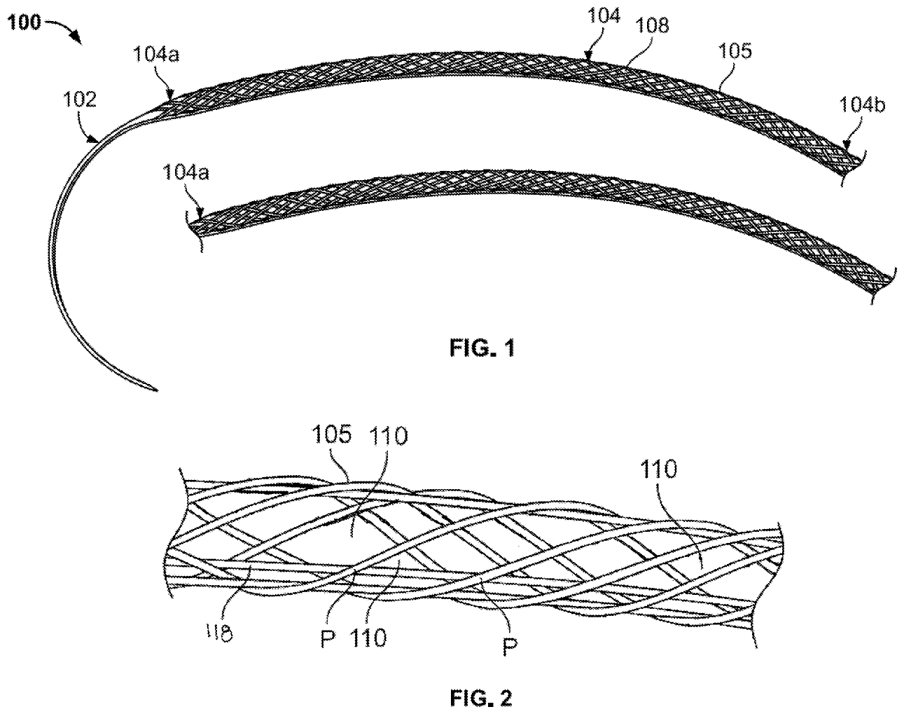

Turning now to FIG. 1, which depicts a medical device 100 constructed in accordance with this disclosure. The medical device 100 includes a surgical needle 102, an elongated suture 104, and a tension indicator. The needle 102 includes a contoured or curved needle with a flattened cross-sectional profile, but needles with generally any geometry could be used. The suture 104 has a first end 104a for being attached to the surgical needle and a second end 104b located away from the first end 104a and the surgical needle 102. The tension indicator is carried by the suture 104 and signals over-tensioning of the suture 104 when a tensile load that exceeds a predetermined tensile load is applied along an axial direction of the suture 104.

As shown in FIG. 1, the entire length of the suture 104 between the first and second ends 104a, 104b can include a tubular wall 106 that defines a hollow core 108. However, less than the entire length of the suture 104 may be tubular. For example, either or both of the first and second ends 104a, 104b can have a non-tubular portion or portion of other geometry. Such non-tubular portions may be for attaching the first end 104a of the suture 104 to the needle 102 or for tying off the second end 104b. In examples where the entire length of the suture 104 is tubular, as shown, the entire length of the suture 104 including the ends and central portion also has a generally constant or uniform diameter or thickness in the absence of stresses. That is, no portion of the suture 104 is meaningfully larger in diameter than any other portion of the suture 104. Moreover, no aspect, end, or other portion of the suture 104 is intended to be or is actually passed through, disposed in, received in, or otherwise positioned inside of the hollow core 108. The hollow core 108 is adapted for receiving tissue in-growth only.

In some examples, the tubular wall 106 has a diameter in a range of approximately 1 mm to approximately 10 mm. The tubular wall 106 can be constructed of a material such as, for example, polyethylene terephthalate, nylon, polyolefin, polypropylene, silk, polymers p-dioxanone, co-polymer of p-dioxanone, ε-caprolactone, glycolide, L(−)-lactide, D(+)-lactide, meso-lactide, trimethylene carbonate, polydioxanone homopolymer, poly-4-hydroxybutyrate, and combinations thereof. The addition of colors to these filaments would not change the overall concept of a suture that communicates with the surgeon. Similarly, the use of barbs on these filaments, some that may “hold” at a certain tension and others that “fail” at a higher tension would be consistent with the teachings of communicative sutures. So constructed, the tubular wall 106 of the suture 104 can be radially deformable such that it adopts a first cross-sectional profile in the absence of lateral stresses and a second cross-sectional profile in the presence of lateral stresses. For example, in the absence of lateral stresses the tubular wall 106 and, therefore the suture 104, can have a circular cross-sectional profile, thereby exhibiting radial symmetry. In the presence of a lateral stress, such a suture 104 could then exhibit a partially or wholly collapsed conformation.

In at least one example of the medical device 100, at least a portion of the tubular wall 106 is macroporous defining a plurality of pores 110 (e.g., openings, apertures, holes, etc.), only a few of which are expressly identified by reference number and lead line in FIG. 1 for clarity. The pores 110 extend completely through the mesh wall 105 to the hollow core 108. In some examples, the tubular wall 106 can be constructed of a woven or knitted mesh material.

As used herein, the term “macroporous” can include pore sizes that are at least greater than or equal to approximately 200 microns and, preferably, greater than or equal to 500 microns. In some examples of the medical device 100, the size of at least some of the pores 110 in the suture 104 are in a range of approximately 500 microns to approximately 4 mm. In another example, at least some of the pores 110 have a pore size in the range of approximately 500 microns to approximately 2.5 mm. In yet another example, at least some of the pores 110 have a pore size in the range of approximately 1 mm to approximately 2.5 mm. In other examples, the size of at least some of the pores 110 are approximately 2 mm.

Moreover, the pores 110 may vary in size. For example, some of the pores 110 can be macroporous (e.g., greater than approximately 200 microns) and some of the pores 110 can be microporous (e.g., less than approximately 200 microns). The presence of microporosity (i.e., pores less than approximately 200 microns) in such examples of the disclosed suture may only be incidental to the manufacturing process, which can including knitting, weaving, extruding, blow molding, or otherwise, but not necessarily intended for any other functional reason regarding biocompatibility or tissue integration. The presence of microporosity (i.e, some pores less than approximately 200 microns in size) as a byproduct or incidental result of manufacturing does not change the character of the disclosed macroporous suture (e.g., with pores greater than approximately 200 microns, and preferably greater than approximately 500 microns, for example), which facilitates tissue in-growth to aid biocompatibility, reduce tissue inflammation, and decrease suture pull-through.

In versions of the disclosed suture that has both macroporosity and microporosity, the number of pores 20 that are macroporous can be in a range from approximately 1% of the pores to approximately 99% of the pores (when measured by pore cross-sectional area), in a range from approximately 5% of the pores to approximately 99% of the pores (when measured by pore cross-sectional area), in a range from approximately 10% to approximately 99% of the pores (when measured by pore cross-sectional area), in a range from approximately 20% of the pores to approximately 99% of the pores (when measured by pore cross-sectional area), in a range from approximately 30% of the pores to approximately 99% of the pores (when measured by pore cross-sectional area), in a range from approximately 50% of the pores to approximately 99% of the pores (when measured by pore cross-sectional area), in a range from approximately 60% of the pores to approximately 99% of the pores (when measured by pore cross-sectional area), in a range from approximately 70% of the pores to approximately 99% of the pores (when measured by pore cross-sectional area), in a range from approximately 80% of the pores to approximately 99% of the pores (when measured by pore cross-sectional area), or in a range from approximately 90% of the pores to approximately 99% of the pores (when measured by pore cross-sectional area).

So configured, the pores 110 in the suture 104 are arranged and configured such that the suture 104 is adapted to facilitate and allow tissue in-growth and integration through the pores 110 in the mesh wall 105 and into the hollow core 108 when introduced into a body. That is, the pores 110 are of sufficient size to achieve maximum biocompatibility by promoting neovascularization and local/normal tissue in-growth through the pores 110 and into the hollow core 108 of the suture 104. As such, tissue growth through the pores 110 and into the hollow core 108 enables the suture 104 and resultant tissue to combine and cooperatively increase the strength and efficacy of the medical device 100, while also decreasing irritation, inflammation, local tissue necrosis, and likelihood of pull through. Instead, the suture 104 promotes the production of healthy new tissue throughout the suture construct including inside the pores 110 and the hollow core 108.

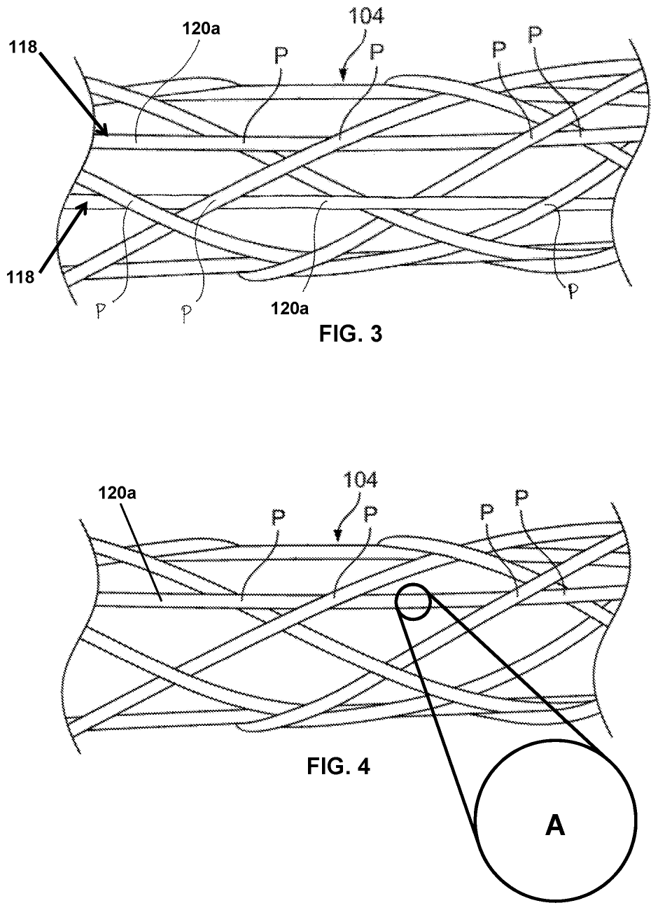

The suture 104 additionally carries the tension indicator 118 as depicted in FIGS. 2-6. The tension indicator 118 signals over-tensioning of the suture 104 when a tensile load is applied to the suture 104 between the first and second ends 104a, 104b. The tension indicator 118 may be in the form of an elongated element, or filament, 120 extending substantially, or entirely, the length of the suture 104 between the first and second ends 104a, 104b. The filament 120 is fixed to the mesh wall 116 at a plurality of points P. Additionally, the filament 120 may be either a solid core filament or a hollow core filament. The hollow core filament, in particular, may have an object disposed within the hollow core of the filament. The tension indicator 118 may signal over-tensioning via tactile alert, visual alert, auditory alarm, or any combination thereof.

For example, the filament 120 can signal over-tensioning through a tactile alert. In particular, the filament 120 may signal over-tensioning by breaking when an applied tensile load exceeds a predetermined tensile load. The predetermined tensile load is less than or equal to a tensile load that, when applied to the suture 104, causes roping of the suture 104. By breaking before the applied tension load reaches the tensile load that causes roping, the tension indicator 118 signals that the suture 104 has been over-tensioned.

In another example, the tension indicator 118 can signal over-tensioning by breaking a first filament 120a when the applied tensile load is greater than a first rupture load and breaking a second filament 120b when the applied tensile load is greater than a second rupture load. The first rupture load may be less than the second rupture load. In some examples, breaking the first filament 120a signals to a user that the applied tensile load has properly tensioned the suture 104. Additionally, breaking the first and the second filament 120a, 120b signals to a user that the applied tensile load has over-tensioned the suture 104. In such an example, the first rupture load is less than the second rupture load and the second rupture load is greater than a predetermined rupture load. The predetermined rupture load is less than or equal to a tensile load that, when applied to the suture 104, causes roping of the suture 104. Thus, a tensile indicator 120 constructed in accordance with this example would provide a user with a first tactile signal (e.g., breaking of the first filament 120a) to indicate that the suture 104 has been adequately tensioned and a second tactile signal (e.g., breaking of the second filament 120b) to indicate that the suture 104 has been over-tensioned.

In some examples, breaking the first and second filaments 120a, 120b can create an audible signal (i.e., a sound) in addition to a tactile alert as described above. In particular, breaking the first and second filaments 120a, 120b may creating a “popping” or “snapping” sound. The first and second filaments 120a, 120b may make the same sound when broken. However, in other examples, the first filament 120a can create a first sound, having a first frequency and first decibel level, when broken and the second filament 120b can create a second sound, having a second frequency and second decibel level, when broken. Breaking of these communicative filaments may or may not change the ultimate tensile strength of the suture.

In other examples, the filament 120 can signal over-tensioning through a visual alert. In particular, the filament 120 may signal over-tensioning by changing colors as a tensile load is applied to the suture 104. In such an example, the filament 120 may change colors in a variety of ways. For example, the filament 120 can exhibit an initial color (e.g., blue) when not under a tensile load and would change to a final color (e.g., red) when an applied tensile load exceeds a predetermined tensile load. For example, U.S. Application No. 2015/0362669 by Aizenberg et al., which is incorporated herein by reference, discloses a tunable band-gap multilayer fiber that changes color in response to varying strain applied to the multilayer fiber, and the entire contents thereof are hereby incorporated herein by reference. The predetermined tensile load can be less than or equal to a tensile load that, when applied, causes roping of the suture 104. The predetermined tensile load can be, for example, zero (0) to ten (10) Newtons (“N”) when using the suture 104 in an abdominal wall. In orthopedics, a greater force may be required. For example, the predetermined tensile load can be zero (0) to forty (40) N or even higher in such settings.

However, the filament 120 may also change colors progressively. In particular, as a greater tensile load is applied to the suture 104 while the suture 104 is being tensioned, the filament 120 may change from the initial color (e.g., blue) to a set of intermediate colors (e.g., green, yellow, orange) before changing to the final color (e.g., red). In such an example, the filament 120 would be the initial color when not under a tensile load and change to the final color when under a tensile load that exceeds the predetermined tensile load, but would change from color to color in the set of intermediate colors as the applied tensile load increases. In particular, the filament 120 may change from the initial color to a first intermediate color when a tensile load is applied. The first intermediate color would, for example, signal to a user that the applied tensile load is in a first tensile load range. If the applied tensile load falls within the first tensile load range, then the color of the filament 120 would change from the initial color to the first intermediate color; the initial color and the first intermediate color being different colors.

If the applied tensile load is greater than the first tensile load range, the color of the filament 120 would then change from the first intermediate color to a second intermediate color. The second intermediate color would then signal to a user that the applied tensile load is in a second tensile load range. The second tensile load range is greater than the first tensile load range, but less than the predetermined tensile load. The second intermediate color is different from the first intermediate color in order to signal a change from the first tensile load range to the second tensile load range. If, however, the applied tensile load becomes greater than the second tensile load range, the color of the filament 120 will change from the second intermediate color to the final color signaling over-tensioning of the suture.

While the first intermediate color corresponding to the first tensile load range and the second intermediate color corresponding to the second tensile load range are disclosed herein, it should not be interpreted that the disclosed tension indicator 118 is limited to only two different colors in the set of intermediate colors. The tension indicator 118 may, in some other examples, have a third intermediate color corresponding to a third tensile load range and, in yet other examples, have a fourth intermediate color corresponding to a fourth tensile load range. The amount of intermediate colors between the initial and final colors may be determined based on the application of the suture 104. Various suture applications require varying amount of tensioning and, thus, the amount of colors in the set of intermediate colors and their respective tensile load ranges may be adjusted depending on the particular application of the suture.

In addition to the filament 120 changing colors detectable by a human eye, some example filaments 120 may require the use of an ultraviolet light (or light in a different wavelength range) for the human eye to detect the change in color of the filament 120. In such examples, an ultraviolet light source (e.g., a Wood's lamp) is shined on the filament 120 during tensioning of the suture. As the suture 104 is tensioned, the ultraviolet light source allows the change in color of the filament to be detected by the human eye. In such an example, the filament 120 can be construed with fluorescent materials that change color as an applied tensile load increases. Using the ultraviolet light source may also provide the added benefit of sterilizing the wound that the suture is applied to as well as the area surrounding the wound.

In other examples, roping itself may be a visual alert that the suture 104 has been over-tensioned. As discussed extensively above, roping occurs when a suture is over-tensioned during wound closure. In particular, as a tensile load is applied that exceeds a roping tensile load, an outer diameter of the suture 104 decreases and the pore size of the suture collapses. The decrease in outer suture diameter and collapse of suture pore size can be visibly apparent and acts as a visual alert to the healthcare professional, for example, that the suture has been over-tensioned. In some examples, when roping occurs, the pore size of the suture 104 collapses permanently and a new suture 104 needs to be used by the surgeon. Such an example suture 104 can visually indicate over-tensioning only once. In other examples, however, the suture 104 is elastic and the decrease in pore size of the suture 104 is temporary. In such an example, as a tensile load is applied along the axial direction of the suture 104, the diameter of the suture changes from a first diameter to a second diameter causing the pore size of the suture 104 to become smaller, which visually indicates over-tensioning of the suture. However, as the applied tensile force is removed, the diameter of the suture 104 changes from the second diameter to the first diameter causing the pores 110 to become larger and return to an original size. The elasticity of the pores 110 allows the suture 104 to visually indicate over-tensioning each time the applied tensile load is greater than the predetermined tensile load that causes roping. Thus, the elasticity of the pores 110 allows the suture 104 to visually indicated over-tensioning multiple times during use, rather than a single time, as is the case with sutures 104 that have pores 110 that permanently deform.

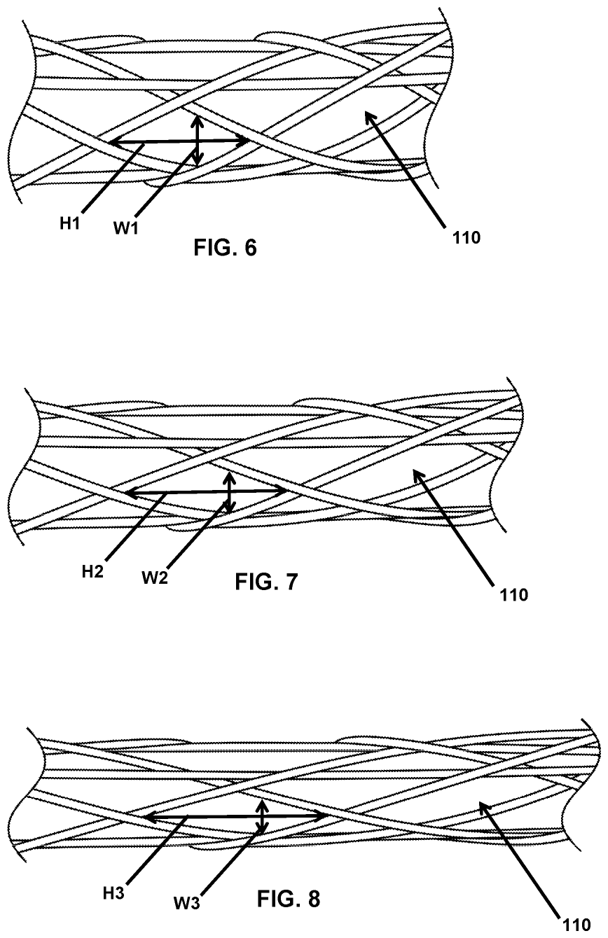

The roping phenomenon as an indication of over-tensioning is best depicted in FIGS. 6-8. FIG. 6 depicts the suture 104 in a resting state (i.e., not under tensile stress) and has pores 110 with a height H1 and a width W1. As the suture 104 is tensioned, the applied tensile stress causes the diameter of the suture 104 to decrease and, in response, the pores 110 of the suture 104 change shape. In particular, as depicted in FIG. 7, the height and width of the pores 110 may change from height H1 and width W1 to height H2 and width W2, respectively. In such an example, height H2 is less than height H1 and width W2 is greater than W1. As the suture is tensioned further, a greater tensile stress is applied to the suture 104, which causes the diameter of the suture 104 to further decrease and, in response, further deformation of the pores 110 occurs. FIG. 8, for example, depicts the further deformation of the pores 110 as greater tensile stress is applied. In particular, the height H2 and width W2 may change to a height H3 and a width W3, where height H3 is less than height H2 and width W3 is greater than width W2.

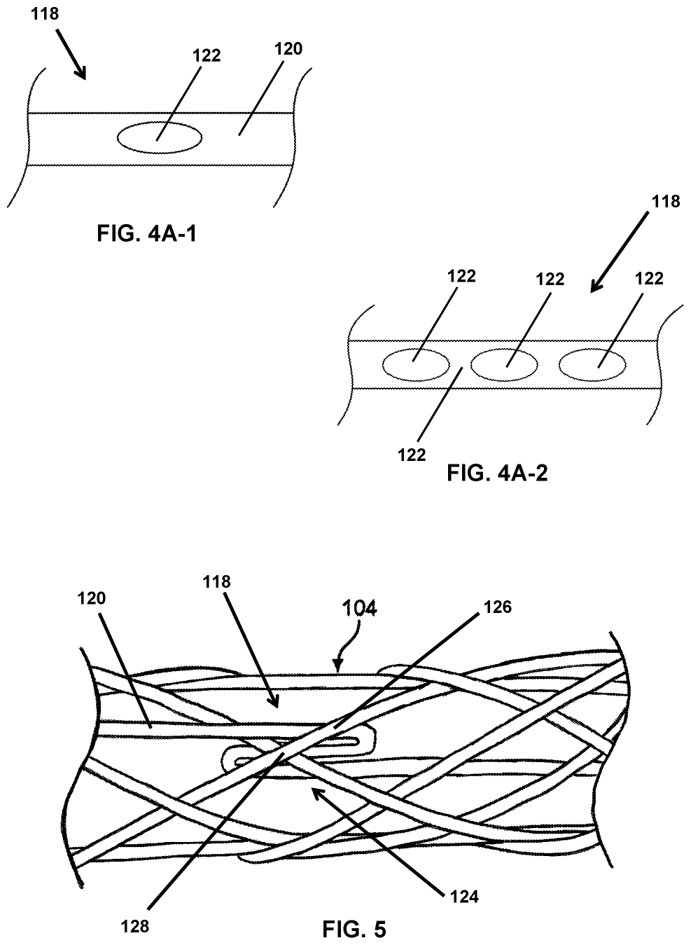

In yet other examples, the filament 120 can signal over-tensioning through an auditory alarm. In particular, the filament 120 may signal over-tensioning by rupturing a capsule 122 disposed within the filament 120 as depicted in FIGS. 4 and 4A. For example, the capsule 122 disposed within the filament 120 can break when the applied tensile load is greater than a predetermined tensile load, which is less than or equal to a tensile load that, when applied, causes roping of the suture 104. When broken, the capsule 122 creates an auditory alarm signaling over-tensioning of the suture 104. To rupture the capsule 122, in some examples, a compressive force is exerted on the capsule 122 as the suture 104 is tensioned. In particular, when a tensile load is applied to the suture 104, the diameter of the filament 120 decreases and exerts a radial compressive force on the capsule 122. When the radial compressive force exerted on the capsule 122 exceeds a capsule rupture load, the capsule 122 ruptures and creates the auditory alarm.

The capsule rupture load may depend on the application of the suture 104. For example, the capsule rupture load may be in the range of 0-40 N. In particular, the capsule rupture load may be less than ten (10) N.

In another example, at least one capsule 122 is disposed within the filament 120 as depicted in FIG. 5. In particular, a first capsule 122a having a first rupture load and a second capsule 122b having a second rupture load may be disposed within the filament 120. In such an example, the second rupture load is greater than the first rupture load and the second rupture load is less than a predetermined rupture load, which is less than or equal to a tensile load that, when applied, causes roping of the suture 104. Thus, as the suture 104 is tensioned and the applied tensile load increases, the first capsule 122a ruptures and creates a first auditory alarm and then the second capsule 122b ruptures and creates a second auditory alarm. In some examples, the first auditory alarm and the second auditory alarm are the same frequency and decibel level. In another example, the first auditory alarm is a first frequency and a first decibel level and the second auditory alarm is a second frequency and a second decibel level.

In such an example, the first auditory alarm signals proper tensioning of the suture 104 and alerts the user to stop tensioning the suture 104. If, however, the second capsule 122b ruptures creating the second auditory alarm, the second auditory alarm would signal over-tensioning of the suture 104.

Moreover, the capsule 122 disposed within the filament 120 may be filled with a fluid. In particular, the capsule 122 can be filled with a liquid that visually alerts a surgeon that the capsule 122 disposed within the filament 120 has been ruptured and, thus, indicating that the suture 104 has been over-tensioned. For example, the capsule 122 can be filled with a colored liquid (e.g., blue) that flow out of the capsule 122 once the capsule is ruptured. In such an example, the colored liquid would flow through the pores of the suture and into the surgical area. Thus, rupturing the capsule 122 filled with liquid creates not only an auditory alarm, but also a visual indication that the suture 104 has been over-tensioned.

Additionally, multiple capsules 122 may be disposed within the filament with each capsule 122 filled with a fluid. In particular, each of the fluid filled capsules may have a particular rupture load, as discussed above, and a distinct liquid disposed within the capsule to provide various visual indications. For example, a first capsule may be filled with a first colored liquid (e.g., blue), the second capsule may be filled with a second colored liquid (e.g., yellow, green, orange), and the third capsule may be filled with a third colored liquid (e.g., red) where the first, second, and third colored liquids are different colors. In such an example, the first capsule has a first rupture load, the second capsule has a second rupture load, and the third capsule has a third rupture load, as discussed above. The first, second, and third rupture loads are distinct and may, for example, be different rupture loads that correspond to various ranges of tensile loads applied during tensioning of the suture. For example, the second rupture load can be greater than the first rupture load, but less than the third rupture load. Thus, as the suture is tensioned, a first, a second, and a third visual indicator may be created when the first, second, and third capsules, respectively, rupture causing the respective colored liquids to flow from the ruptured capsule 122.

While the forgoing capsules 122 were discussed as being filled with a liquid, the capsules 122 may be filled with another fluid. For example, the capsules 122 may be filled with a gas such that, when the capsule 122 is ruptured, an olfactory alert is created, alerting the surgeon the suture has been over-tensioned. In yet other examples, the capsules 122 may be filled with a powder that changes to a fluid when in contact with mucosa in the area the suture is being placed.

In addition to being filled with a fluid to signal over-tensioning, the capsules 122 may also be filled with a medication. For example, the capsule 122 can be filled with a medication to assist in the healing process. In particular, the medication may be an antiseptic, an antibiotic, a nonsteroidal anti-inflammatory drugs (“NSAID”), or a pain relief medication. The medication may, in other examples, be a topical anesthetic. In yet other examples, the medication may be a fluid that helps to alleviate agitation of the mucosa, submucosa, or muscle tissue surrounding the suture site.

The tension indicator 118 may also be tuned to a variety of tensile loads depending on the particular application of the suture 104. In particular, each tension indicator 118 may be manufactured to a particular set of tensile requirements depending on the commercial application of a suture carrying the tension indicator 118. For example, the tension indicator 118 can be tuned to a lower tensile load if the application of the suture 104 requires a lesser tensile load applied when tensioning the suture to close a wound. In such an example, a lesser tensile load would have to be applied before the tension indicator 118 signaled over-tensioning. However, in other examples, the tension indicator 118 can be tuned to a greater tensile load if the application of the suture requires a greater tensile load when tensioning the suture to close a wound. In such an example, a greater tensile load would have to be applied before the tension indicator 118 signaled over-tensioning.

Tuning the tension indicator 118 can include varying the size of the filament 120 in some examples. In other examples, the tension indicator 118 is tuned by varying the material used to construct the filament 120. In yet other examples, tuning the tension indicator 118 includes varying the dimensions of the filament 120 and the material used to construct the filament 120. By varying the material used to construct the filament 120 and the dimensions of the filament 120 during production, the tension indicator may be dialed in to various ranges of tension depending on the application of the suture.

In yet another example, the filament 120 is fixed to the mesh wall 116 at a fixed area 124 between the first and second ends 104a, 104b of the suture 104. The fixed area 124 includes a first point 126 fixedly attaching the filament 120 to the mesh wall 116 and a second point 128 releasably attaching the filament 120 to the mesh wall 116. The first point 126 is disposed toward the second end 104b of the suture 104 and the second point 128 is disposed toward the first end 104a of the suture 104. The second point 128 breaks as the suture 104 is tensioned creating an audible alarm signaling over-tensioning of the suture 104.

In particular, the second point 128 has a rupture load that is less than a predetermined rupture load. The predetermined rupture load is less than or equal to a tensile load that, when applied to the suture 104, causes roping of the suture 104. When the applied tensile load to the suture 104 is greater than the rupture load of the second point 128, the second point 128 breaks and the filament 120 is then only attached to the mesh wall 116 at the first point in the fixed area 124. Breaking the second point 128 creates the auditory alarm signaling over-tensioning of the suture 104.

While the suture 104 in FIG. 1 has been described as including a single elongated hollow core 108, in some embodiments, a suture according to the present disclosure can comprise a tubular wall defining a hollow core including one or more interior voids (e.g., extending the length of the suture). In some versions, at least some of the interior voids can have a size or diameter >approximately 200 microns, >approximately 300 microns, >approximately 400 microns, >approximately 500 microns, >approximately 600 microns, >approximately 700 microns, >approximately 800 microns, >approximately 900 microns, >approximately 1 millimeter, or >approximately 2 millimeters.

Additionally, a suture according to the present disclosure may include a tubular wall defining a hollow core including one or more lumens (e.g., running the length of the suture). In some examples, the tubular wall defining the hollow core can include a honeycomb structure, a 3D lattice structure, or other suitable interior matrix, which defines the one or more interior lumens. In some examples, at least some of the interior lumens in the honeycomb structure, 3D lattice structure, or other suitable matrix can have a size or diameter >approximately 200 microns, >approximately 300 microns, >approximately 400 microns, >approximately 500 microns, >approximately 600 microns, >approximately 700 microns, >approximately 800 microns, >approximately 900 microns, >approximately 1 millimeter, or >approximately 2 millimeters. In some examples, a hollow core can include a hollow cylindrical space in the tubular wall, but as described, the term “hollow core” is not limited to defining a cylindrical space, but rather could include a labyrinth of interior voids defined by a honeycomb structure, a 3D lattice structure, or some other suitable matrix.

The sutures of the present disclosure may also include a hollow, flexible structure that has a circular cross-sectional profile in its non-stressed state, but which collapses into a more flattened cross-sectional shape when pulled in an off-axis direction. In some examples, sutures are provided that exhibit radial symmetry in a non-stressed state. The radial symmetry in a non-stressed state may eliminate the need for directional orientation while suturing. In other examples, sutures are provided that exhibit a flattened cross-sectional profile when off-axis (longitudinal axis) force is applied (e.g., tightening of the suture against tissue), thereby more evenly distributing the force applied by the suture on the tissue. Additionally, in some examples, the sutures can include a flexible structure that adopts a first cross-sectional profile in its non-stressed state (e.g., suturing profile), but adopts a second cross-sectional profile when pulled in an off-axis direction (e.g., tightened profile).

In other examples, the internal voids are configured to encourage the suture to adopt a preferred conformation when in a stressed state. The internal voids may be configured, for example, to encourage the suture to adopt a broadened leading edge to displace pressure across the contacted tissue when in a tightened profile. In some other examples, internal voids are configured to allow a suture to adopt radial exterior symmetry (e.g., circular outer cross-sectional profile) when in a non-stressed state. In yet other examples, varying the size, shape, and/or placement of internal voids alters one or both of the first cross-sectional profile (e.g., non-stressed profile, suturing profile) and second cross-sectional profile (e.g., off-axis profile, stressed profile, tightened profile).

Sutures, which are substantially linear in geometry, have two distinct ends, as described above with reference to FIG. 1, for example. In some examples, both ends are identical. In other examples, each end is different. In yet other examples, one or both ends are structurally unadorned. The one or more ends may be attached to or at least configured for attachment to a needle via swaging, sonic welding, adhesive, tying, or some other means (as shown FIG. 1). In other examples, the second end 104b of the suture 104 is configured to include an anchor for anchoring the suture 104 against the tissue through which the suture 104 is inserted. In some examples, the second end 104b of the suture 104 is configured to anchor the suture at the beginning of the closure. In other examples, the second end 104b of the suture 104 includes an anchor which has a structure that prevents the suture 104 from being pulled completely through the tissue. The anchor has a greater dimension than the rest of the suture 104 to prevent the suture from being completely pulled through the tissue. Generally, the anchor includes a structure with any suitable shape for preventing the suture 104 from being pulled through the hole. For example, the anchor may take the shape of a ball, disc, plate, or cylinder.

In other examples, the anchor of the suture 104 includes a closed loop. In such examples, the closed loop is of any suitable structure including, but not limited to a crimpled loop, flattened loop, or a formed loop. The loop can be integrated into the end of the suture 104, in some examples, or a separate loop structure may be attached to the suture 104. Additionally, the needle 102 may be passed through the closed loop anchor to create a cinch for anchoring the suture 14 to that point. In some examples, the anchor 22 can include one or more structures (e.g., barb, hook, etc.) to hold the end of the suture 14 in place. One or more anchor 22 structures (e.g., barb, hook, etc.) may be used in conjunction with a closed loop to ratchet down the cinch and hold its position. In yet other examples, a knotless anchoring system can be provided.

The present disclosure provides suturing needles with cross-sectional profiles configured to prevent suture pull-through and methods of use thereof. In particular, suturing needles may have cross-sectional shapes that reduce tension against the tissue at the puncture site and reduce the likelihood of tissue tear. For example, the suturing needles can have a flat, elliptical, transitioning over the length of the needle, etc. cross-sectional shape. In some examples, one cross-sectional dimension of the needle is greater than the orthogonal cross-sectional dimension. Additionally, the suturing needles may have a circular shape at its point (e.g., distal end), but transition to a flattened profile (e.g., ribbon-like) at the rear (e.g. proximal end). The face of the flattened area may be orthogonal to the radius of curvature of the needle. Suturing needles may also create a slit (or flat puncture) in the tissue as it is passed through, rather than a circle or point puncture. In such examples, suturing needles have a circular shape at its point (e.g., distal end), but transition to a two-dimensional (2D) cross-sectional profile (e.g., ellipse, crescent, half moon, gibbous, etc.) at the rear (e.g. proximal end).

The surface and/or internal texture of the disclosed suture 104 may promote tissue adhesion and/or ingrowth. In particular, the suture may include a porous (e.g., macroporous) and/or textured material, which allows tissue adhesion and/or ingrowth. The pores of the porous material may take the form of various shapes depending on the application of the suture. For example, the porous suture may be a two-dimensional (2D) cross-sectional profile (e.g., elliptical, circular (e.g., collapsible circle), half moon, crescent, concave ribbon, etc.). The porous material may be constructed from polypropylene or any other suitable suture material.

The suture may also include any surface texture suitable to promote tissue in-growth and/or adhesion. Suitable surface textures include, for example, ribbing, webbing, mesh, grooves, etc. Additionally, the suture may include filaments or other structures to provide increased surface area and/or increased stability of suture within tissue. Finally, interconnected porous architecture may be provided, in which pore size, porosity, pore shape and/or pore alignment facilitates tissue in-growth.

The suture may also include a mesh and/or mesh-like exterior. In particular, the mesh exterior provides a flexible suture that spreads pressure across the closure site and allows for significant tissue in-growth. The density of the mesh may be tailored to obtain desired flexibility, elasticity, and in-growth characteristics.

In some examples, the suture is coated and/or embedded with materials to promote tissue ingrowth. Examples of biologically active compounds that may be used in sutures to promote tissue ingrowth include, but are not limited to, cell attachment mediators, such as the peptide containing variations of the “RGD” integrin binding sequence known to affect cellular attachment, biologically active ligands, and substances that enhance or exclude particular varieties of cellular or tissue ingrowth. Such substances include, for example, osteoinductive substances, such as bone morphogenic proteins (BMP), epidermal growth factor (EGF), fibroblast growth factor (FGF), platelet-derived growth factor (PDGF), insulin-like growth factor (IGF-I and II), TGF-β, etc. Additional examples of pharmaceutically active compounds that may be used in sutures to promote tissue ingrowth include, but are not limited to, acyclovir, cephradine, malfalen, procaine, ephedrine, adriomycin, daunomycin, plumbagin, atropine, guanine, digoxin, quinidine, biologically active peptides, chlorin e.sub.6, cephalothin, proline and proline analogues such as cis-hydroxy-L-proline, penicillin V, aspirin, ibuprofen, steroids, nicotinic acid, chemodeoxycholic acid, chlorambucil, and the like. Therapeutically effective dosages may be determined by either in vitro or in vivo methods.

Sutures may have braided or monofilament constructions. In particular, some sutures are provided in single-armed or double-armed configurations with a surgical needle mounted to one or both ends of the suture, or may be provided without surgical needles mounted. In some examples, the suture include one or more biocompatible materials such as one or more bioabsorbable and nonabsorbable materials. For example, the sutures may include one or more aromatic polyesters such as polyethylene terephthalate, nylons such as nylon 6 and nylon 66, polyolefins such as polypropylene, silk, and other nonabsorbable polymers. The sutures may also include one or more polymers and/or copolymers of p-dioxanone (also known as 1,4-dioxane-2-one), c-caprolactone, glycolide, L(−)-lactide, D(+)-lactide, meso-lactide, trimethylene carbonate, and combinations thereof. In some other examples, the sutures comprise polydioxanone homopolymer. Any suitable suture materials or combinations thereof are within the scope of the present disclosure, including poly-4-hydroxybutyrate.

Additionally, the sutures may include sterile, medical grade, surgical grade, and or biodegradable materials. For example, the suture 104 is coated with, contains, and/or elutes one or more bioactive substances (e.g., antiseptic, antibiotic, anesthetic, promoter of healing, etc.).

Additionally, the structure and material of the suture may provide physiologically-tuned elasticity. In particular, a suture of appropriate elasticity is selected for a tissue. For example, in some examples, sutures for use in abdominal wall closure will have similar elasticity to the abdominal wall, so as to reversibly deform along with the abdominal wall, rather than act as a relatively rigid structure that would carry higher risk of pull-through. In other examples, elasticity would not be so great however, so as to form a loose closure that could easily be pulled apart. For example, deformation of the suture would start occurring just before the elastic limit of its surrounding tissue, e.g., before the tissue starts tearing or irreversibly deforming.

The sutures described herein may provide a suitable replacement or alternative for surgical repair meshes (e.g., those used in hernia repair). In particular, the use of sutures in place of mesh reduces the amount of foreign material placed into a subject (e.g., 50 cm² (suture) v. 240 cm² (mesh)). Further, the decreased likelihood of suture pull-through allows the use of sutures to close tissues not possible with traditional sutures (e.g., areas of poor tissue quality (e.g., friable or weak tissue) due to conditions like inflammation, fibrosis, atrophy, denervation, congenital disorders, attenuation due to age, or other acute and chronic diseases). Like a surgical mesh, sutures described herein permit a distribution of forces over a larger area thereby delocalizing forces felt by the tissue and reducing the chance of suture pull-though and failure of the closure.

The disclosed sutures may be permanent, removable, or absorbable. Permanent sutures provide added strength to a closure or other region of the body, without the expectation that the sutures will be removed upon the tissue obtaining sufficient strength. In such examples, materials are selected that pose little risk of long-term residency in a tissue or body. Removable sutures are stable (e.g., do not readily degrade in a physiological environment), and are intended for removal when the surrounding tissue reaches full closure strength. Absorbable sutures integrate with the tissue in the same manner as permanent or removable sutures, but eventually biodegrade and/or are absorbed into the tissue after having served the utility of holding the tissue together during the post-operative and/or healing period. Use of absorbable sutures may present a reduced foreign body risk.

Although prevention of dehiscence of abdominal closures (e.g., hernia formation) is specifically described in the present disclosure, the sutures described herein are useful for joining any tissue types throughout the body. The sutures described herein are of particular utility to closures that are subject to tension and/or for which cheesewiring is a concern. Exemplary tissues within which the present disclosure finds use include, but are not limited to: connective tissue, muscle, dermal tissue, cartilage, tendon, or any other soft tissues. Specific applications of sutures described herein include placation, suspensions, slings, etc. Sutures described herein find use in surgical procedures, non-surgical medical procedures, veterinary procedures, in-field medical procedures, etc. The scope of the present disclosure is not limited by the potential applications of the sutures described herein.

Yet, from the foregoing, it should also be appreciated that the present disclosure additionally provides both a method of re-apposing soft tissue and a method of manufacturing a medical device.

While mesh sutures were primarily discussed throughout the disclosure, the tension indicator disclosed may be used in various other types of sutures. For example, the tension indicator can be used with monofilament sutures, braided sutures, or a suture constructed of a mesh. The aforementioned is not an exhaustive list, but merely an exemplary list of various sutures.

Based on the present disclosure, a method of signaling over-tensioning of a suture 104 having a tension indicator 118, the tension indicator 118 including a filament 120, carried by the suture 104 can first include piercing a portion of soft tissue with a surgical needle attached to a first end 104a of the suture 104. Then threading the suture 104 through the soft tissue and make one or more stitches. Finally, applying a tensile load along an axial direction of the suture 104 until the tension indicator 118 signals over-tensioning of the suture 104. Signaling over-tensioning of the suture 104 may include creating a tactile alert, a visual alert, or an auditory alarm as discussed above. For example, signaling over-tensioning via a tactile alert can include breaking the filament 112 as discussed above. In another example, signaling over-tensioning via a visual alert may include changing the color of the filament 118 as described in detail above. Finally, signaling over-tensioning via an auditory alarm may include breaking a capsule 122 disposed within the filament 118 as discussed in detail above. Additionally, signaling over-tensioning via auditory alarm can include breaking a second point 128 that releasably attaches the filament 120 to the mesh wall 116. When broken, the second point 128 creates an audible noise.

A method of manufacturing a medical device in accordance with the present disclosure can include forming a tubular wall 116 having a plurality or pores 110 and defining a hollow core 108, each pore 110 having a pore size that is greater than or equal to approximately 500 microns. Additionally, the method of manufacturing can include attaching a first end 104a of the tubular wall 104 to a surgical needle 102. Forming the tubular wall 104 can include forming a tube from a mesh material. The tubular mesh wall 116 may be formed by directly weaving or knitting fibers into a tube shape. Alternatively, forming the tubular mesh wall 116 can include weaving or knitting fibers into a planar sheet and subsequently forming the planar sheet into a tube shape. Of course, other manufacturing possibilities exist and knitting and weaving fibers are not the only possibilities for creating a porous tube within the scope of the present disclosure, but rather, are mere examples.

We claim:

1. A medical device comprising:
an elongated suture having a first end adapted to be attached to a surgical needle and a second end located away from the first end; and
a tension indicator carried by the elongated suture, the tension indicator signaling over-tensioning of the elongated suture when a tensile load that exceeds a predetermined tensile load is applied along an axial direction between the first and second ends of the elongated suture.

2. The medical device of claim 1, wherein the elongated suture is one of (a) a monofilament suture, (b) a braided suture, or (c) a mesh suture.

3. The medical device of claim 1, wherein the tension indicator comprises a filament having a length dimension extending substantially entirely from the first end to the second end of the elongated suture.

4. The medical device of claim 3, wherein the filament breaks when the applied tensile load exceeds the predetermined tensile load.

5. The medical device of claim 4, wherein the predetermined tensile load is less than a roping tensile load which, when applied along the axial direction of the elongated suture, causes roping of the elongated suture.

6. The medical device of claim 3, wherein the filament changes color when the applied tensile load exceeds the predetermined tensile load.

7. The medical device of claim 4, wherein the predetermined tensile load is less than a tensile load which, when applied along the axial direction of the elongated suture, causes roping of the elongated suture.

8. The medical device of claim 7, wherein the color of the filament changes from an initial color to a final color when the applied tensile load exceeds the predetermined tensile load, the final color signaling over-tensioning of the elongated suture.

9. The medical device of claim 8, wherein the color of the filament changes from the initial color to a first intermediate color when the applied tensile load is less than the predetermined tensile load.

10. The medical device of claim 9, wherein the color of the filament changes from the first intermediate color to a second intermediate color when the applied tensile load is equal to or approximately equal to the predetermined tensile load.

11. The medical device of claim 10, wherein the color of the filament changes from the second intermediate color to the final color when the applied tensile load is greater than the predetermined tensile load.

12. The medical device of claim 10, wherein the initial color, the first intermediate color, the second intermediate color, and the final color are different colors.

13. The medical device of claim 3, wherein the filament activates an auditory alarm to generate an auditory signal when the applied tensile load exceeds the predetermined tensile load.

14. The medical device of claim 13, wherein the predetermined tensile load is less than a roping tensile load which, when applied along the axial direction of the elongated suture, causes roping of the elongated suture.

15. The medical device of claim 14, wherein the auditory alarm comprises a capsule disposed within the filament, wherein the capsule ruptures when the applied tensile load exceeds a capsule rupture load creating the auditory signal.

16. The medical device of claim 15, wherein the capsule rupture load is greater than the predetermined tensile load.

17. The medical device of claim 15, wherein a diameter of the filament decreases as the applied tensile load increases, the decrease in the filament diameter exerting a compressive radial force on the capsule until the capsule ruptures.

18. The medical device of claim 14, wherein the auditory alarm comprises a first capsule having a first rupture load, a second capsule having a second rupture load, and a third capsule having a third rupture load disposed within the filament, wherein the first, second, and third capsules rupture when the applied tensile load is greater than the first, second, and third rupture loads, respectively, creating the auditory signal.

19. The medical device of claim 18, wherein the third rupture load is greater than the second rupture load and the second rupture load is greater than the first rupture load.

20. The medical device of claim 18, wherein the first capsule creates a first auditory signal having a first frequency and a first decibel level when ruptured, the second capsule creates a second auditory signal having a second frequency and second decibel level when ruptured, and the third capsule creates a third auditory signal having a third frequency and a third decibel level when ruptured.

21. The medical device of claim 20, wherein the third frequency is higher than the second frequency and the second frequency is higher than the first frequency; and
the third decibel level is greater than the second decibel level and the second decibel level is greater than the first decibel level.

22. The medical device of claim 14, wherein the auditory alarm comprises a fixed area between the first and second ends of the elongated suture, the fixed area including a first point fixedly attaching the filament to the elongated suture and a second point releasably attaching the filament to the elongated suture.

23. The medical device of claim 22, wherein the first point is disposed toward the second end of the elongated suture and the second point is disposed toward the first end of the elongated suture.

24. The medical device of claim 22, wherein the applied tensile load breaks the second point, creating an audible signal, when the applied tensile load is greater than the predetermined tensile load.

25. The medical device of claim 1, wherein the tension indicator comprises a first filament having a length dimension extending substantially entirely from the first end to the second end of the elongated suture and a second filament having a length dimension extending substantially entirely from the first end to the second end of the elongated suture, the first filament having a first rupture load and the second filament having a second rupture load.

26. The medical device of claim 25, wherein the first filament ruptures when the applied tensile load exceeds the first rupture load and the second filament ruptures when the applied tensile load exceeds the second rupture load.

27. The medical device of claim 26, wherein the first rupture load is less than the second rupture load and the second rupture load is greater than the predetermined tensile load.

28. The medical device of claim 27, wherein the predetermined tensile load is less than a roping tensile load which, when applied along the axial direction of the elongated suture, causes roping of the elongated suture.

29. The medical device of claim 28, wherein the elongated suture further comprises:
a flat wall or a hollow tubular wall and a plurality of pores extending through the flat wall, at least some of the pores having a pore size that is greater than or equal to approximately 200 microns such that the pores are adapted to facilitate tissue integration through the mesh wall when the elongated mesh suture is introduced into a body.

30. The medical device of claim 29, wherein the elongated suture comprises a first diameter when the tensile load is not applied along the axial direction between the first and second ends of the elongated suture and a second diameter when the tensile load is applied along the axial direction between the first and second ends of the elongated suture, and wherein the elongated sutures changes from the second diameter to the first diameter when the tensile load is removed.

31. The medical device of claim 30, wherein the first diameter comprises the plurality of pores having a first size and the second diameter comprises the plurality of pores having a second size different from the first size.

32. A method of signaling over-tensioning of an elongated suture comprising a tension indicator carried by the elongated suture, the method comprising:
piercing a portion of soft tissue with a surgical needle attached to a first end of the elongated suture;
threading the elongated suture through the soft tissue, wherein the elongated suture includes the first end and a second end located away from the surgical needle;
applying a tensile load along an axial direction of the elongated suture between the first and second ends of the elongated suture; and
signaling, via the tension indicator, over-tensioning of the elongated suture.

33. The method of claim 32, wherein signaling over-tensioning of the elongated suture comprises breaking a filament of the suture when the applied tensile load exceeds a first rupture load equating to a predetermined tensile load.

34. The method of claim 32, wherein signaling over-tensioning of the elongated suture comprises changing the color of a filament of the suture when the applied tensile load exceeds a predetermined tensile load.

35. The method of claim 34, wherein signaling over-tensioning of the elongated suture comprises changing the color of a filament of the suture from an initial color to a final color when the applied tensile load exceeds the predetermined tensile load, the initial color being different than the final color.

36. The method of claim 35, wherein signaling over-tensioning of the elongated suture further comprises changing the color of the filament from the initial color to a first intermediate color when the applied tensile load is less than the predetermined tensile load, the initial color being different than the first intermediate color.

37. The method of claim 36, wherein signaling over-tensioning of the elongated suture further comprises changing the color of the filament from the first intermediate color to a second intermediate color when the applied tensile load is equal to or approximately equal to the predetermined tensile load, the first intermediate color being different than the second intermediate color.

38. The method of claim 37, wherein signaling over-tensioning of the elongated suture further comprises changing the color of the filament from the second intermediate color to the final color when the applied tensile load exceeds the predetermined tensile load, the second intermediate color being different from the final color.

39. The method of claim 32, wherein signaling over-tensioning of the elongated suture comprises alerting a user via auditory signal when the applied tensile load exceeds a predetermined tensile load.

40. The method of claim 39, wherein signaling over-tensioning of the elongated suture comprises rupturing a capsule disposed within the filament, the capsule rupturing when the applied tensile load exceeds a capsule rupture load creating the auditory signal, and wherein the capsule rupture load is greater than the predetermined tensile load.

41. The method of claim 40, wherein signaling over-tensioning of the elongated suture comprises applying a tensile load decreasing the filament diameter, which exerts a radial force on the capsule, until the capsule ruptures.

42. The method of claim 39, wherein signaling over-tensioning of the elongated suture comprises continuously applying a tensile load to the elongated suture, such that a first capsule disposed within the filament ruptures at a first rupture load, a second capsule disposed within the filament ruptures at a second rupture load, and a third capsule disposed within the filament ruptures at a third rupture load, wherein the third rupture load is greater than the second rupture load and the second rupture load is greater than the first rupture load.

43. The method of claim 39, wherein signaling over-tensioning of the elongated suture comprises breaking a second fixed point when the applied tensile load is greater than the predetermined tensile load, the second fixed point disposed proximate a first fixed point, the second fixed point disposed toward the first end of the suture, the first fixed point disposed toward the second end of the suture, the second fixed point releasably attaching the filament to the elongated suture, and the first fixed point fixedly attaching the filament to the elongated suture.

44. The method of claim 33, wherein signaling over-tensioning of the elongated suture comprises breaking the filament when the applied tensile load exceeds the first rupture load and breaking a second filament carried by the elongated suture and having a second rupture load when the applied tensile load exceeds the second rupture load, wherein the first rupture load is less than the second rupture load, and the second rupture load is less than a predetermined tensile load, which, when applied along the axial direction of the elongated suture, causes roping of the elongated suture.

45. The method of claim 31, wherein applying the tensile load along the axial direction of the elongated suture between the first and second ends of the elongated suture changes the diameter of the elongated suture from a first diameter to a second diameter, and wherein removing the tensile load changes the diameter of the elongated suture from the second diameter to the first diameter.
